(19) United States
(12) Patent Application Publication
(10) Pub. No.: US 2012/0289487 A1
(43) Pub. Date: Nov. 15, 2012

(54) Pharmaceutical Use of Multicyclic Compounds as Anti-Aids Agents

(75) Inventors: Luiz Francisco Pianowski, Braganca Paulista (BR); Joao Batista Calixto, Florianopolis (BR); Claudio Paulino Chaves, Fortaleza (BR)

(73) Assignee: AMAZÔNIA FITOMEDICAMENTOS LTDA, Fortaleza CE (BR)

(21) Appl. No.: 13/522,385

(22) PCT Filed: Jan. 15, 2010
(86) PCT No.: PCT/IB2010/050198

§ 371 Date: Jul. 16, 2012

(51) Int. Cl. A61K 31/575; A61P 31/18; A61K 31/22

(52) U.S. Cl. 514/170; 514/511; 514/182; 514/171

(57) ABSTRACT

This invention refers to pharmaceutical use of multicyclic compounds chosen from ingenols, lanosta-8,24-dien-3-ols and their mixtures, as anti-AIDS agents.

This invention generally refers to a pharmaceutical use of multicyclic compounds, namely ingenol and lanosta-8,24-dien-3-ol compounds and their mixtures, as anti-AIDS agents.

### BACKGROUND

Acquired immune deficiency syndrome (also known AIDS) is a disease of the human immune system caused by the human immunodeficiency virus (HIV).

This syndrome progressively reduces the effectiveness of the immune system and leaves individuals susceptible to opportunistic infections and tumors. HIV is transmitted through direct contact of a mucous membrane or the bloodstream with a bodily fluid containing the virus, such as blood, semen, vaginal fluid, preseminal fluid, and breast milk.

AIDS is now pandemic. According to health organizations, in 2007, it was estimated that 33.2 million people lived with the disease worldwide, and that AIDS killed an estimated 2.1 million people, including 330,000 children.

The pathophysiology of AIDS is complex, as is the case with all syndromes. HIV is a retrovirus that primarily infects vital organs of the human immune system such as CD4+ T cells (a subset of T cells), macrophages and dendritic cells. It directly and indirectly destroys CD4+ T cells. T lymphocytes are essential to the immune response and without them the body cannot fight infections or kill cancerous cells. This weakens the immune system and allows opportunistic infections.

Although there are treatments for AIDS that can slow the course of the disease, there is currently no publicly available vaccine for HIV or cure for HIV or AIDS. The only known methods of prevention are based on avoiding exposure to the virus or, failing that, an antiretroviral treatment directly after a highly significant exposure, called post-exposure prophylaxis (PEP).

Antiretroviral treatment reduces both the mortality and the morbidity of HIV infection, but these drugs are expensive and routine access to antiretroviral medication is not available in all countries. It also has very unpleasant side effects including diarrhea, malaise, nausea and fatigue. In the absence of antiretroviral therapy, the average time of progression from HIV infection to AIDS is almost ten years, depending on the HIV subtype, and the average survival time after developing AIDS is only a few months.

Current treatment for HIV infection consists of highly active antiretroviral therapy, in general, comprising combinations (or “cocktails”) consisting of at least three drugs belonging to at least two types of antiretroviral agents.

Non-adherence and non-persistence with therapy are the major reasons why some people do not benefit from antiretroviral therapy. The reasons for non-adherence and non-persistence are varied. Major psychosocial issues include poor access to medical care, inadequate social supports, psychiatric disease and drug abuse. Antiretroviral therapy regimens can also be complex and thus hard to follow, with large numbers of pills taken frequently. Side effects can also deter people from persisting with antiretroviral therapy, these include lipodystrophy, dyslipidaemia, diarrhea, insulin resistance, an increase in cardiovascular risks and birth defects.

Since 1987, chronic administration of AZT and similar nucleoside analogs has been prescribed to AIDS patients to inhibit human immunodeficiency virus (HIV) as the most common treatment. Since 1990, AZT has also been prescribed to healthy HIV antibody-positive persons to prevent AIDS. The rationale is to inhibit HIV DNA synthesis at doses that do not inhibit cell DNA synthesis (Chiu et al.: The toxicity of azidothymidine (AZT) on human and animal cells in culture at concentrations used for antiviral therapy, Genetica 95: 103-109, 1995).

However, in view of its inherent cytotoxicity and hematologic toxicity, AZT has been questioned as an acceptable anti-HIV drug (Chiu et al. mentioned above and Clotet et at.: Toxicity of Zidovudine (AZT) in patients with AIDS. Int Conf AIDS 4-9; 5: 338 1989).

Several independent studies reported that AZT is toxic to human cells in culture, i.e. that the half inhibitory doses (ID 50) ranged between 1 and 50 μM. In accordance with these results, life threatening toxicity including anemia, leukopenia, nausea, muscle atrophy, dementia, hepatitis and mortality, has been documented in humans treated with 20 to 60 μM AZT, i.e. the advisability of AZT as an anti-HIV drug can be reconsidered (Chiu et al. mentioned above).

Research to improve current treatments includes decreasing side effects of current drugs, further simplifying drug regimens to improve adherence, and determining the best sequence of regimens to manage drug resistance.

### THE INVENTION

The present invention refers to the use of ingenol and lanosta-8,24-dien-3-ol compounds, and their mixtures, or compositions therewith, for the effective treatment of AIDS significantly without the drawbacks known up to now, i.e. related to toxicity.

Without excluding any other, adequate ingenol compounds are one or more of 3-(2,4,6-dodecatrienoyl)-ingenol, 3-(2,4,6,8-tetradecatetranoyl)-ingenol, pharmaceutically acceptable salts thereof, isomers, polymorphs, solvates or hydrates, prodrugs or metabolites thereof.

Ingenol compounds can be obtained for instance from Euphorbiaceae plants, or by chemical synthesis, the path being irrelevant to the invention. Adequate ingenol compounds may, for instance, be provided as a fraction of a polar solvent extract of an Euphorbiaceae plant latex according to international patent publication WO 2007000618.

Without excluding any other, adequate lanosta-8,24-dien-3-ols are one or more of euphol (RN 514-47-6), tirucallol (RN 514-46-5) and lanosterol (RN 79-63-0), pharmaceutically acceptable salts thereof, isomers, crystals and polymorphs, solvates and hydrates, prodrugs or metabolites thereof. Lanosta-8,24-dien-3-ols can be obtained for instance from Euphorbiaceae plants, or by chemical synthesis, the path being irrelevant to the invention.

Adequate mixtures of ingenols and lanosta-8,24-dien-3-ols range from 1:100 to 100:1, particularly 1:50 to 50:1, more particularly 1:10 to 10:1, more particularly 1:4 to 4:1.

Therefore, in a first aspect, the invention concerns the use of ingenol and/or lanosta-8,24-dien-3-ol compounds for the production of pharmaceutical compositions that inhibit the human immunodeficiency virus replication, i.e. useful in the treatment of HIV infections, AIDS.

The ingenol and lanosta-8,24-dien-3-ols compounds of the invention, their mixtures and compositions comprising them according to the invention, can be administered to the subject in need of treatment in any adequate way, enteral or parenteral, including oral, topical, transdermal, subcutaneous, intraperitonial, intravenous, by infiltration, by inhalation, transdermal, transmucosal, intramuscular, intrapulmonary, vaginal, rectal, intraocular, and sublingual. Particularly adequate ways of administration in the present invention are topically and systemically (infiltration, oral, inhalation by spray, transdermal). The ingenol compounds of the invention can be comprised in slow or controlled release compositions. Known adjuvants and excipients can be utilized in ingenol compounds containing compositions—a reference for pharmaceutical dosage forms useful for the compositions related to the invention can be found in the publication Remington's Pharmaceutical Sciences, Mack Publishing, 1965-1990.

The compositions of the invention can be administered to patients as solids, liquids or semi-liquids, tablets, capsules, pills, powder, granules, suspensions, emulsions, dispersions and any other useful known pharmaceutically acceptable form. The compositions might contain further active agents, for instance antibiotics, depending on the desired effect. For oral administration as tablets or capsules (both soft and hard capsules), the ingenol compounds can be combined with pharmaceutically acceptable inert vehicles, such as lactose, starch, sucrose, glucose, methyl cellulose, magnesium stearate, dicalcium phosphate, calcium phosphate, mannitol, sorbitol, and similars; for oral administration in the liquid form, the ingenol compounds can be combined with ethanol, glycerol, water, and similars. When desired or necessary, agglomerating agents, lubricant agents, disintegrating agents, color and fragrance can be added to the mixture. Common agglomerating agents are glucose, [beta]-lactose, corn sweeteners, natural or synthetic gums such as gum arabica, tragacanth or sodium alginate, carboxymethylcellulose, polyethylene glycol, wax and similars. Lubricants include sodium oleate, sodium stearate, magnesium stearate, sodium benzoate, sodium acetate, sodium chloride. Disintegrants include starch, methyl cellulose, agar, bentonite, xanthan gum, and similars.

The compositions concerned in the invention can also be administrated as liposomes or coupled with soluble polymers as vehicles.

Liquid dosage forms for oral administration may comprise colorants and edulcorants to increase acceptance by patients. Acceptable vehicles for water dosage forms are, water, an appropriate oil, a saline solution, aqueous dextrose, other sugar solutions and glycols as propylene glycol or polyethylene glycols, phosphate buffer.

In another aspect, the invention concerns a method to inhibit the human immunodeficiency virus replication, i.e. for the treatment of human immunodeficiency virus infections, such as AIDS, said method comprising the administration to a patient of a pharmacological effective amount of the compounds according to the present invention in a pharmacologically acceptable carrier. A example of adequate amount of one or more ingenol or one or more lanosta-8,24-dien-3-ols, or their mixtures, corresponds to a dosage of 0.001 to 2000 mg per kg of patient weight of one or more ingenols, or one or more lanosta-8,24-dien-3-ols, or their mixtures, administered to such a patient one or more times a day.

### EXAMPLES

Though the following examples are concrete embodiments of the invention, they do not in any way impose limitations to it, other than what is expressed in the claims presented further on. In the text that follows, the compound euphol, a member of the lanosta-8,24-dien-3-ol family, will often be mentioned, and it is to be understood that this is done simply for ease of reference, and no other lanosta-8,24-dien-3-ol compound is, for this reason, excluded from the invention.

### Virus and Cells

Mononuclear cells of peripheral blood (MCPB) were obtained from healthy donors by the Ficol-Hipaque gradient of density method (Hystopaque, Chem Sigma. Co., USA). Those cells were re-suspended in RPMI 1640 medium (source: Sigma-Aldrich, USA)) supplemented with 10% inactivated bovine fetal serum (source: HyClone, USA), penicillin (100 U/ml), streptomycin (100 μg/ml), 2 mM glutamine, stimulated with 5 μg/ml of phytohemagglutinin (PHA, source: Sigma-Aldrich, USA) for 2 to 3 days, washed and again kept in a culture medium containing 5 U/ml of human recombinant interleukin-2 (source: Sigma Aldrich, USA). MCPB (3×10⁶) were distributed in plates with 48 wells in RPMI medium without serum for 1 hour in atmosphere of 5% CO₂ at 37° C. Primary HIV-1 isolated with distinct tropisms for chemokine receptors, namely CCR5-tropic (R5-tropic) were used.

### Tests of Cellular Viability (XTT and Blue Trypan)

The effect of the isolates upon cellular viability was evaluated by the XTT method (2,3-bis[2-methoxy-4-nitro-5-sulphophenyl]2H-tetrazolium-carboxanilide, source: Sigma). In order to test the effect of the compounds on the mitocondrial activity, MCPB of normal individuals (2×105 cells/200 μl/well), in plates with 96 wells, 37° C., 5% CO2) were subjected to different concentrations of the substances (0.5 μg/mL, 5 μg/mL, 20 μg/mL, 40 μg/mL, 80 μg/mL, 160 μg/mL, 320 μg/mL and 640 μg/mL) during 3 to 7 days. At the end of this period, the cellular activity was determined by colorimetry by the addition of 50 ml of the XTT solution, and the results were analyzed by the reading of absorbance at 450 nm (according to Scudiero et al, 1988, Cancer Res. 48:4827-4833).

### Anti-HIV-1 Inhibitory Activity

### MCPB

MCPB were infected with R5 isolates, using 5 to 10 ng/ml of AG p24 HIV-1 for 2 to 3 hours. The cells were washed 3 times, re-suspended in culture medium supplemented with 5 μ/ml of IL-2 and plated in plates of 96 wells (2×10⁵ cells/200 μl in triplicate, and treated with the concentrations indicated for the tested compounds (0.5 μg/mL, 1.0 μg/mL, 5.0 μg/mL, 10 μg/mL, 20 μg/mL and 40 μg/mL). The cultures were kept at 37° C. in 5% CO₂ atmosphere for seven days and the viral response was measured by the dosage of p24 by ELISA (Enzyme Linked ImmunoSorbent Assay Information).

### Result of the Substance Tests

### Citotoxicity and Antiretroviral Assays

The analyses were carried out in mononuclear Cells of peripheral blood (MCPB), wherein the cytotoxicity assays were evaluated by XTT and the assays of inhibition of the viral response were evaluated by ELISA.

CC50 (50% cytotoxic concentration) indicates the concentration of the tested substance able to keep viable 50% of the cells.

EC50 (50% effective concentration) is concentration of the substance able to inhibit 50% of the viral particle production.

### Example 1

### Citotoxicity of Ingenols

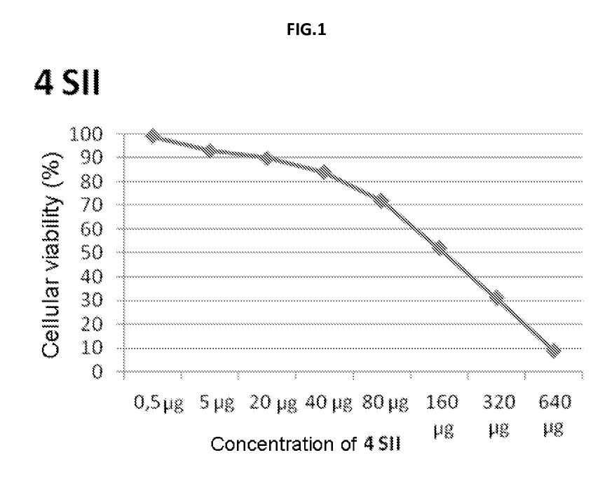

The results of table I below, also shown in FIG. 1, relate to a 1:1 mixture of 3-(2,4,6-dodecatrienoyl)-ingenol and 3-(2,4,6,8-tetradecatetranoyl)-ingenol, from a fraction of a polar solvent extract of an Euphorbiaceae plant latex, as described in international patent publication WO 2007000618. Lab Code: 4Sll

TABLE I

| Concentration | Viability |
|---|---|
| μg/ml | % |
| 0.5 | 99 |
| 5 | 93 |
| 20 | 90 |
| 40 | 84 |
| 80 | 72 |
| 160 | 52 |
| 320 | 31 |
| 640 | 9 |

CC50 = 167 μg

### Example 2

### Inhibition of Viral Response of Ingenols

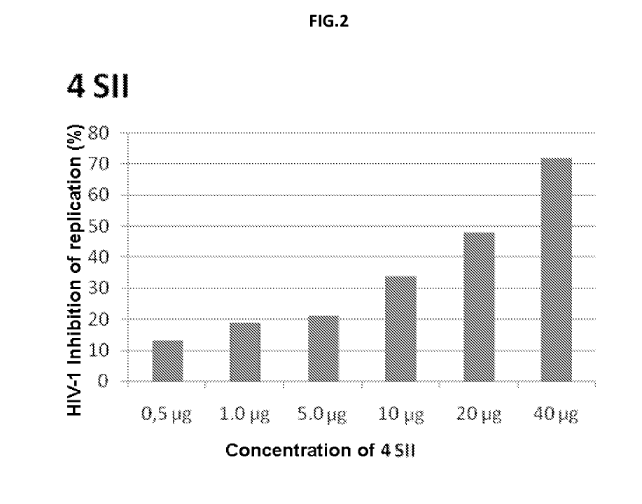

The results of table II, also shown in FIG. 2, relate to a 1:1 mixture of 3-(2,4,6-dodecatrienoyl)-ingenol and 3-(2,4,6,8-tetradecatetranoyl)-ingenol, from an active fraction of a polar solvent extract of an Euphorbiaceae plant latex as described in international patent publication WO 2007000618. Lab Code: 4Sll

TABLE II

| Concentration | Viability |
|---|---|
| μg/ml | % |
| 0.5 | 13 |
| 1.0 | 19 |
| 5.0 | 21 |
| 10 | 34 |
| 20 | 48 |
| 40 | 72 |

EC50 = 28 μg/ml

### Example 3

### Citotoxicity of Lanosta-8,24-dien-3-ols

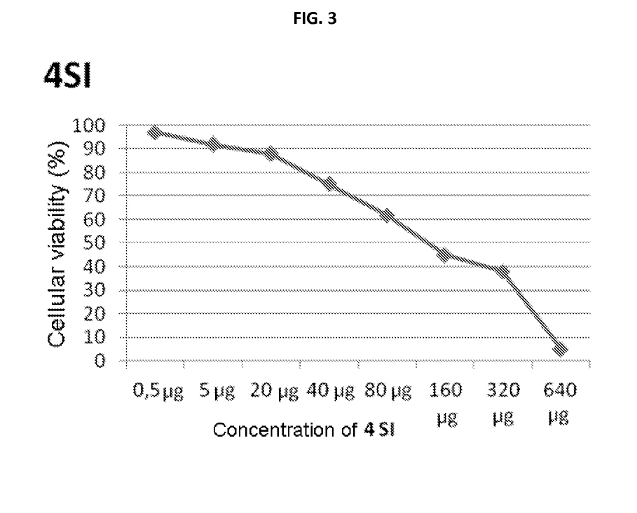

The results of table III, also shown in FIG. 3, relate to a mixture of about 92% euphol and 8% tirucallol. Lab Code: 4Sl.

TABLE III

| Concentration | Viability |
|---|---|
| μg/ml | % |
| 0.5 | 97 |
| 5 | 92 |
| 20 | 88 |
| 40 | 75 |
| 80 | 62 |
| 160 | 45 |
| 320 | 38 |
| 640 | 5 |

CC50 = 138 μg/ml

### Example 4

### Inhibition of Viral Response of Lanosta-8,24-dien-3-ols

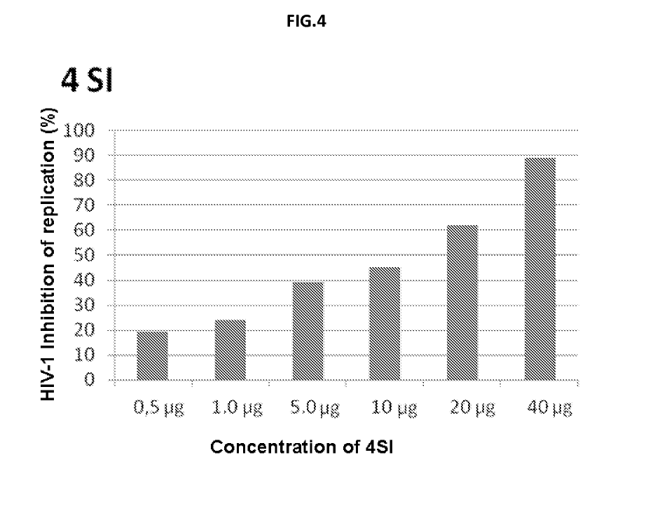

The results of table IV, also shown in FIG. 4, relate to a mixture of about 92% euphol and 8% tirucallol—Lab Code: 4Sl

TABLE IV

| Concentration | Inhibition |
|---|---|
| μg/ml | % |
| 0.5 | 19 |
| 1.0 | 24 |
| 5.0 | 39 |
| 10 | 45 |
| 20 | 62 |
| 40 | 89 |

EC50 = 16 μg

### Example 5

### Citotoxicity of Mixtures of Ingenols and Lanosta-8,24-dien-3-ols

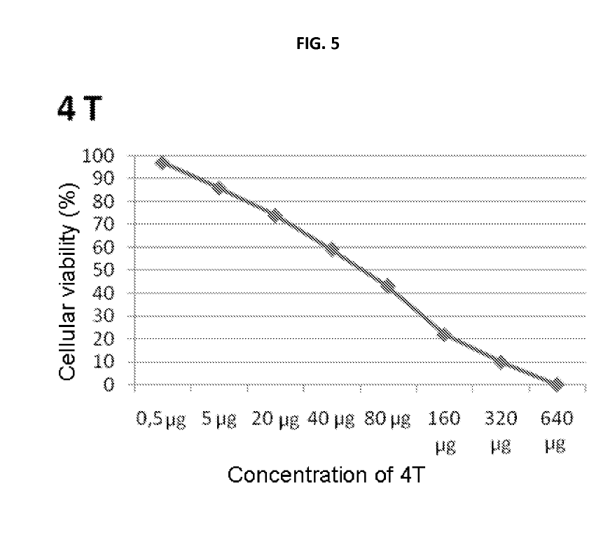

The results of table V, also shown in FIG. 5, relate to a butanol extract of Euphorbia tirucalli latex, comprising approximately 70% euphol, 10% tirucallol, 10% 3-(2,4,6-dodecatrienoyl)-ingenol and 10% 3-(2,4,6,8-tetradecatetranoyl)-ingenol (such percentages take into account only the compounds of the invention themselves, not the remaining of the extract). Lab Code 4T.

TABLE V

| Concentration | Viability |
|---|---|
| μg/ml | % |
| 0.5 | 97 |
| 5 | 86 |
| 20 | 74 |
| 40 | 59 |
| 80 | 43 |
| 160 | 22 |
| 320 | 10 |
| 640 | 0 |

CC50 = 44 μg/ml

### Example 6

### Inhibition of Viral Response of Mixtures of Ingenols and Lanosta-8,24-dien-3-ols

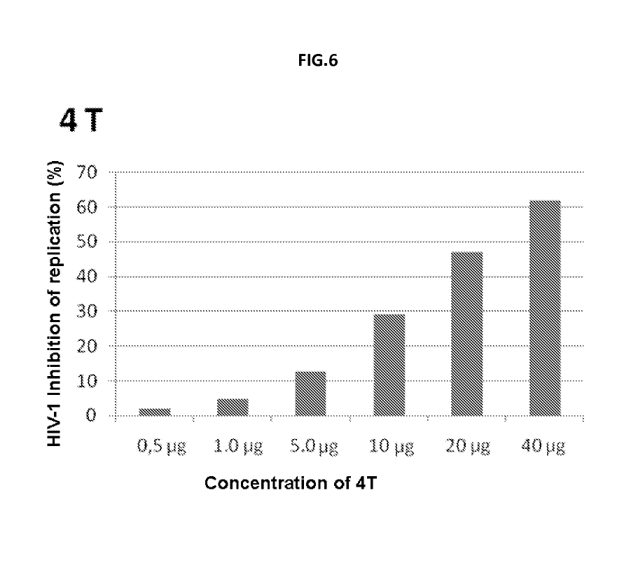

The results of table VI, also shown in FIG. 6, relate to a polar solvent extract of Euphorbia tirucalli latex, comprising approximately 70% euphol, 10% tirucallol, 10% 3-(2,4,6-dodecatrienoyl)-ingenol and 10% 3-(2,4,6,8-tetradecatetranoyl)-ingenol (such percentages take into account only the compounds of the invention themselves, not the remaining of the extract). Lab Code 4T.

TABLE VI

| Concentration | Inhibition |
|---|---|
| μg/ml | % |
| 0.5 | 2 |
| 1.0 | 4.8 |
| 5.0 | 12.5 |
| 10 | 29 |
| 20 | 47 |
| 40 | 62 |

EC50 = 31 μg/ml

Description of the extract made from Euphorbia tirucalli extract, utilized in examples 5 and 6: to fresh latex diluted 1:1 in water, hexane is added, to obtain coagulation. The insolubilized material is washed several times with water and filtered. The obtained material is submitted to a mixture of butanol and hexane, the more polar substances remaining in the butanol, which is then separated from the hexane, filtrated and finally withdrawn by evaporation.

### Comments

TABLE VII
below summarizes the results of examples 1 to 6 above.

| | Cellular | % viral | | Viral |
|---|---|---|---|---|
| | viability | replication | | replication |
| | CC50 | inhibition | SI | inhibition by |
| ESSAY | μg/ml | EC50 μg/ml | (CC50/EC50) | 40 μ/mL (%) |
| Lanosta- | 138 | 16 | 8.6 | 8.6 |
| 8,24-dien- | | | | |
| 3-ols (4SI) | | | | |
| Ingenols | 167 | 28 | 5.9 | 5.9 |
| (4SII) | | | | |
| Mixture of | 44 | 31 | 1.4 | 1.4 |
| Lanosta- | | | | |
| 8,24-dien- | | | | |
| 3-ols and | | | | |
| ingenols | | | | |
| (4T) | | | | |

SI (selectivity index) = CC50/EC50, represents the degree of security in the use of a drug, therefore the higher the value, the better the effect of the substance is.

### Analysis of the Results

The results of the examples above show that the compounds of the invention are capable of inhibiting the production of HIV-1 in mononuclear cells of peripheral blood (MCPB) evaluated by the ELIZA P24 method.

The higher toxicity seen in the mixture of ingenols and lanosta-8,24-dien-3-ols is not unexpected, as they were not isolated from the about 30% remaining components of the extract of the Euphorbia latex.

But it can be clearly seen that the compounds of the invention and their mixtures inhibited the production of HIV, with limited toxicity.

It is well understood that, with the help of the teachings and examples presented herein, a person skilled in the art is able to reproduce the invention in equivalent ways, using the same functions to obtain similar results, without departing from the scope of the invention defined in the attached claims.

1. A pharmaceutical composition for the prevention or treatment of HIV infections, said composition comprising multicyclic compounds selected from the group consisting of: ingenols, lanosta-8,24-dien-3-ols and mixtures thereof; in combination with a pharmaceutically acceptable vehicle.

2. The composition according to claim 1, wherein said ingenol compounds are selected from the group consisting of: 3-(2,4,6-dodecatrienoyl)-ingenol, 3-(2,4,6,8-tetradecatetranoyl)-ingenol, pharmaceutically acceptable salts thereof, isomers, polymorphs, solvates or hydrates, prodrugs or metabolites thereof, and mixtures thereof.

3. The composition according to claim 1, wherein said lanosta-8,24-dien-3-ols compounds are selected from the group consisting of: euphol, tirucal lol and lanosterol, pharmaceutically acceptable salts thereof, isomers, crystals and polymorphs, solvates and hydrates, prodrugs or metabolites thereof, and mixtures thereof.

4. The composition according to claim 1, wherein the mixtures of ingenols and lanosta-8,24-dien-3-ols are in an amount in a range from 1:100 to 100:1.

5. Method for the treatment of HIV infections, wherein said method comprises the administration to a patient of a pharmacological effective amount of ingenol compounds in a pharmacologically acceptable carrier.

6. The method according to claim 5, wherein said ingenol compounds are selected from the group consisting of: 3-(2,4,6-dodecatrienoyl)-ingenol, 3-(2,4,6,8-tetradecatetranoyl)-ingenol, pharmaceutically acceptable salts thereof, isomers, crystals and polymorphs, solvates and hydrates, prodrugs or metabolites thereof, and mixtures thereof.

7. Method for the treatment of HIV infections, wherein said method comprises the administration to a patient of a pharmacological effective amount of lanosta-8,24-dien-3-ols compounds in a pharmacologically acceptable carrier.

8. The method according to claim 7 wherein said lanosta-8,24-dien-3-ols are selected from the group consisting of: euphol, tirucal lol and lanosterol, pharmaceutically acceptable salts thereof, isomers, crystals and polymorphs, solvates and hydrates, prodrugs or metabolites thereof, and mixtures thereof.

9. Method for the treatment of HIV infections wherein said method comprises the administration to a patient of a pharmacological effective amount of mixtures of ingenol and lanosta-8,24-dien-3-ol compounds in a pharmacologically acceptable carrier.

10. The method according to claim 9 wherein said mixture of ingenol and lanosta-8,24-dien-3-ol compounds comprises: (A) ingenol compounds selected from the group consisting of: 3 (2,4,6-dodecatrienoyl)-ingenol, 3-(2,4,6,8-tetradecatetranoyl)-ingenol, pharmaceutically acceptable salts thereof, isomers, crystals and polymorphs, solvates and hydrates, prodrugs and metabolites thereof, and mixtures thereof; and (B) lanosta-8,24-dien-3-ols selected from the group consisting of: euphol, tirucallol and lanosterol, pharmaceutically acceptable salts thereof, isomers, crystals and polymorphs, solvates and hydrates, prodrugs and metabolites thereof, and mixtures thereof; wherein the ratio between A and B are in a range from 1:100 to 100:1.

11. The method according to claim 9, wherein said mixture comprises about 70% eufol, 10% tirucallol, 10% 3(2,4,6-dodecatrienoyl)-ingenol and 10% 3-(2,4,6,8-tetradecatetranoyl)-ingenol, in weight.

12. The method, according to claim 9, wherein said effective amount of one or more ingenols, lanosta-8,24-dien-3-ols or their mixture, is 0.001 to 2000 mg per kg of patient weight, administered to such a patient one or more times a day.

13. The composition according to claim 1, wherein the mixtures of ingenols and lanosta-8,24-dien-3-ols are in an amount in a range from 1:50 to 50:1.

14. The composition according to claim 1, wherein the mixtures of ingenols and lanosta-8,24-dien-3-ols are in an amount in a range from 1:10 to 10:1.

15. The composition according to claim 1, wherein the mixtures of ingenols and lanosta-8,24-dien-3-ols are in an amount in a range from 1:4 to 4:1.

16. The method according to claim 9 wherein said mixture of ingenol and lanosta-8,24-dien-3-ol compounds comprises: (A) ingenol compounds selected from the group consisting of: 3-(2,4,6-dodecatrienoyl)-ingenol, 3-(2,4,6,8-tetradecatetranoyl)-ingenol, pharmaceutically acceptable salts thereof, isomers, crystals and polymorphs, solvates and hydrates, prodrugs and metabolites thereof, and mixtures thereof; and (B) lanosta-8,24-dien-3-ols are from the group consisting of: euphol, tirucallol and lanosterol, pharmaceutically acceptable salts thereof, isomers, crystals and polymorphs, solvates and hydrates, prodrugs and metabolites thereof, and mixtures thereof; wherein the ratio between A and B are in a range from 1:50 to 50:1.

17. The method according to claim 9 wherein said mixture of ingenol and lanosta-8,24-dien-3-ol compounds comprises: (A) ingenol compounds selected from the group consisting of: 3-(2,4,6-dodecatrienoyl)-ingenol, 3-(2,4,6,8-tetradecatetranoyl)-ingenol, pharmaceutically acceptable salts thereof, isomers, crystals and polymorphs, solvates and hydrates, prodrugs and metabolites thereof, and mixtures thereof; and (B) lanosta-8,24-dien-3-ols selected from the group consisting of: euphol, tirucallol and lanosterol, pharmaceutically acceptable salts thereof, isomers, crystals and polymorphs, solvates and hydrates, prodrugs and metabolites thereof, and mixtures thereof; wherein the ratio between A and B are in a range from 1:10 to 10:1.

18. The method according to claim 9 wherein said mixture of ingenol and lanosta-8,24-dien-3-ol compounds comprises: (A) ingenol compounds selected from the group consisting of: 3-(2,4,6-dodecatrienoyl)-ingenol, 3-(2,4,6,8-tetradecatetranoyl)-ingenol, pharmaceutically acceptable salts thereof, isomers, crystals and polymorphs, solvates and hydrates, prodrugs and metabolites thereof, and mixtures thereof; and (B) lanosta-8,24-dien-3-ols are from the group consisting of: euphol, tirucallol and lanosterol, pharmaceutically acceptable salts thereof, isomers, crystals and polymorphs, solvates and hydrates, prodrugs and metabolites thereof, and mixtures thereof; wherein the ratio between A and B are in a range from 1:4 to 4:1.
